(19) United States
(12) Patent Application Publication
(10) Pub. No.: US 2025/0255967 A9
(43) Pub. Date: Aug. 14, 2025

(54) NOVEL USE OF POLYMER COMBINATION

(71) Applicant: Reckitt Benckiser Health Limited, Slough, Berkshire (GB)

(72) Inventors: Laurence Arthur HUNT, Hull (GB); Lucy Amber PHILLIPS, Hull (GB); Philip SMITH, Hull (GB); Sophie LEVESLEY, Hull (GB)

(21) Appl. No.: 18/577,534

(22) PCT Filed: Jul. 8, 2022
(86) PCT No.: PCT/GB2022/051758

§ 371 Date: Jan. 8, 2024

(30) Foreign Application Priority Data
Jul. 9, 2021 (GB) 2109932.0

(51) Int. Cl. A61K 47/32; A61K 9/00; A61K 9/14; A61K 31/192

(52) U.S. Cl. CPC A61K 47/32; A61K 9/0056; A61K 9/146; A61K 31/192

(57) ABSTRACT

The use of a combination of a first polymer and a second polymer as a taste-masking agent for an active pharmaceutical ingredient in an amorphous form and pharmaceutically acceptable salts or esters thereof wherein the active pharmaceutical agent is selected from NSAIDs wherein the active pharmaceutical ingredient and the combination of the first polymer and the second polymer are in the form of a solidified melt extrudate.

The present invention is directed to the use of a combination of polymers to improve the palatability of active pharmaceutical ingredients. In particular, the present invention is directed to the use of a combination of polymers to improve the palatability of an NSAID. A preferred NSAID is amorphous ibuprofen.

There are several routes for the administration of active pharmaceutical ingredients (APIs) to a patient, e.g. orally, parenterally, nasally and transdermally. The oral route is an easy and convenient route and as such remains the most attractive for delivery of APIs. The common oral dosage forms are solutions, suspensions, tablets, caplets, liquid filled gelatin capsules, lozenges and troches.

Many APIs have an unpleasant or bitter taste which can impact on their acceptability in some oral dosage forms. It is well known that these unpleasant or bitter taste characteristics limit their consumer acceptability in direct to mouth formats, for example orally dissolving tablets, lyophilisates, granules and liquids.

Conventional techniques for masking the unpleasant or bitter taste of APIs include the addition of flavours and/or sweeteners to the composition, coating the API with substances which prevent it from contacting the taste buds during oral administration.

In addition, a large excess of taste-masking agent is required to prevent or in some way mitigate the bitter taste of any API that is retained in the oral cavity after the composition has passed from there into the gastro-intestinal tract.

Fast acting forms of APIs with high solubility are known to be particularly challenging to taste mask, and conventional effective methods for masking the unpleasant taste characteristics (for example coatings) often impact directly the release of the API itself, thus negatively impacting the favourable pharmacokinetic characteristics and rendering a fast acting, highly soluble API slower releasing and reducing its performance potential.

Recent advances in technology have presented viable dosage alternatives to taste-mask bitter drugs. Several approaches have been reported which involve complexation, freeze-drying, microencapsulation, fluidized-bed coating and supercritical fluids for taste-masking purposes.

A particular problem exists for compositions that are intended to dissolve or disperse in the oral cavity, such as orodispersible tablets or films.

Many APIs do exist in these formats but they often contain taste masking technology which is either ineffective at taste masking the API itself, or they impact the performance of the API itself.

It would, therefore, be desirable to develop a composition which delivers taste masked, fast acting forms of APIs, without impacting the potential for in-vivo release and absorption.

According to a first aspect of the present invention there is provided the use of a combination of a first polymer and a second polymer as a taste-masking agent for an active pharmaceutical ingredient in an amorphous form and pharmaceutically acceptable salts or esters thereof wherein the active pharmaceutical agent can be selected from NSAIDs wherein the active pharmaceutical ingredient and the combination of the first polymer and the second polymer are in the form of a solidified melt extrudate.

The NSAID can be selected from ibuprofen, flurbiprofen, ketoprofen, diclofenac, naproxen, aspirin, indomethacin, and meloxicam. A preferred NSAID is ibuprofen.

The extrudate can comprise up to 50% by weight of the active pharmaceutical ingredient. The extrudate can comprise up to 45% by weight of the active pharmaceutical ingredient. The extrudate can comprise up to 40% by weight of the active pharmaceutical ingredient.

The extrudate can comprise at least 10% by weight of the active pharmaceutical ingredient. The extrudate can comprise at least 15% by weight of the active pharmaceutical ingredient. The extrudate can comprise at least 20% by weight of the active pharmaceutical ingredient. The extrudate can comprise at least 25% by weight of the active pharmaceutical ingredient. The extrudate can comprise at least 30% by weight of the active pharmaceutical ingredient.

The extrudate can comprise from 10% by weight to 50% by weight of the active pharmaceutical ingredient. The extrudate can comprise from 15% by weight to 45% by weight of the active pharmaceutical ingredient. The extrudate can comprise from 15% by weight to 40% by weight of the active pharmaceutical ingredient. The extrudate can comprise from 20% by weight to 40% by weight of the active pharmaceutical ingredient. The extrudate can comprise from 20% by weight to 30% by weight of the active pharmaceutical ingredient. The extrudate can comprise about 20% by weight of the active pharmaceutical ingredient.

In an alternative embodiment, the extrudate can comprise from 30% by weight to 40% NSAID by weight of the active pharmaceutical ingredient.

Typically, the combination of polymers is miscible with the active pharmaceutical ingredient.

The first polymer of the combination of at least two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 30° C. and 60° C. The polymer can have a glass transition temperature of between 40° C. and 50° C.

The first polymer is selected such that it provides stability for the amorphous form of the active pharmaceutical ingredient for a period of at least six months.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months.

The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

The first polymer of the combination of two polymers can be present at a level 30-90% by weight of the extrudate. Preferably the first polymer of the combination of two polymers can be present at a level 40-80% by weight of the extrudate. More preferably, the first polymer of the combination of two polymers can be present at a level 50-70% by weight of the extrudate. Most preferably, the first polymer of the combination of two polymers can be present at a level of about 60% by weight of the extrudate.

The second polymer of the combination of two polymers can be selected from polymers having a glass transition temperature of at least 80° C. The polymer can have a glass transition temperature of at least 90° C. The polymer can have a glass transition temperature of less than 120° C. The polymer can have a glass transition temperature of less than 140° C. The polymer can have a glass transition temperature of between 80° C. and 140° C. The polymer can have a glass transition temperature of between 90° C. and 110° C.

Preferably, the solidified melt extrudate comprises ibuprofen in an amorphous form, a combination of two polymers wherein the first polymer can have a glass transition temperature of between 30° C. and 60° C. and the second polymer can have a glass transition temperature of between 80° C. and 140° C. More preferably, the solidified melt extrudate comprises ibuprofen in an amorphous form, a combination of two polymers wherein the first polymer can have a glass transition temperature of between 40° C. and 50° C. and the second polymer can have a glass transition temperature of between 90° C. and 110° C.

The solidified melt extrudate can consist essentially of ibuprofen in an amorphous form, a combination of two polymers wherein the first polymer can have a glass transition temperature of between 30° C. and 60° C. and the second polymer can have a glass transition temperature of between 80° C. and 140° C.

The solidified melt extrudate can consist essentially of ibuprofen in an amorphous form, a combination of two polymers wherein the first polymer can have a glass transition temperature of between 40° C. and 50° C. and the second polymer can have a glass transition temperature of between 90° C. and 110° C.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64). Alternative preferred polymers can be polyvinylpyrrolidone 12 PF or polyvinylpyrrolidone 17 PF.

Alternatively, the second polymer can be selected from a combination of polymers with the proviso that the combination is selected such that the extrudate has a tensile strength of at least 3N/mm². The second polymer can be a combination of polymers selected from polyvinylpyrrolidone, polyvinylpyrrolidone-vinyl acetate co-polymer and polyvinyl caprolactam-polyvinyl acetate-polyethylene glycol graft copolymer.

The second polymer is selected such that the extrudate has a tensile strength of 5-50 N/mm².

The second polymer of the combination of two polymers can be present in a level of 1-30% by weight of the extrudate. Preferably, the second polymer of the combination of two polymers can be present in a level of 10-25% by weight of the extrudate. More preferably, the second polymer of the combination of two polymers can be present in a level of 15-25% by weight of the extrudate. Most preferably, the second polymer of the combination of two polymers can be present in a level of about 20% by weight of the extrudate.

A preferred combination of the first and second polymers is dimethylaminoethyl methacrylate co-polymer and polyvinylpyrrolidone-vinyl acetate co-polymer (PVPVA64). An alternative preferred combination of the first and second polymers is dimethylaminoethyl methacrylate co-polymer and polyvinylpyrrolidone K12.

The ratio of the first polymer and the second polymer of the combination of two polymers can be selected to be from 10:1 to 1:10. A preferred ratio is from 4:1 to 1:4. A more preferred ratio is from 1:1 to 1:3.

The ratio of the of the active pharmaceutical ingredient, the first polymer and the second polymer can be from 1:1:1 to 1:4:4. Preferably, the ratio of the active pharmaceutical ingredient, the first polymer and the second polymer can be from 1:1.5:1.5 to 1:2.5:2.5. Alternatively, the ratio of the active pharmaceutical ingredient, the first polymer and the second polymer can be from 1:2:1 to 1:4:1.

The active pharmaceutical ingredient is portioned between the each of the first and second polymer of the extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the extrudate. More preferably, from 25% by weight to 40% by weight of the active pharmaceutical ingredient is in the first polymer of the extrudate. Most preferably, from 30% by weight to 35% by weight of the active pharmaceutical ingredient is in the first polymer of the extrudate.

The composition can include one or more excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid, acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone and croscarmellose sodium.

The surfactants can be selected from polysorbates. A preferred surfactant is polysorbate 80 (Tween 80). Polysorbate 80 can be present as a level of 1-3 w/w %.

One or more processing aids such as talc, magnesium silicate and glyceryl monostearate can be added prior to melt extrusion or post-melt extrusion.

One or more additional processing aids can be added post-melt extrusion selected from microcrystalline cellulose, crospovidone, carrageenan, chitosan, pectinic acid, glycerides, beta-cyclodextrin and cellulose derivatives, sorbitol and mannitol and isomers thereof.

The combination of polymers is soluble at a pH of about 1 to about 5. Preferably, the combination of polymers is soluble at a pH of about 2 to about 4.

Preferably the difference in Hansen solubility parameter between the active pharmaceutical ingredient and one or both of the first polymer and the second polymer is less than 10 MPa^{1/2}. More preferably the difference in Hansen solubility parameter between the active pharmaceutical ingredient and one or both of the first polymer and the second polymer is less than 7 MPa^{1/2}.

The extrudate is typically prepared using a hot melt extrusion process.

The melt extrusion process is preferably conducted at a temperature of between 80° C. and 110° C. More preferably, the temperature of the extrusion process is between 90° C. and 100° C. Most preferably, the temperature of the extrusion process is between 93° C. and 98° C.

The screw speed of the extruder can be selected from 1 revolution per minute (rpm) to 30 revolutions per minute (rpm). Preferably the screw speed of the extruder is from 5-20 rpm. More preferably the screw speed of the extruder is about 10 rpm.

The melt extrusion process can be carried out at a preferred temperature from 80° C. to 110° C. and at a preferred extruder screw speed from 1 rpm to 30 rpm. More preferably, the melt extrusion temperature can be between 90° C. and 100° C. and the extruder screw speed can be from 5 rpm to 20 rpm. Most preferably, the melt extrusion temperature can be between 93° C. and 98° C. and the extruder screw speed can be about 10 rpm.

Alternatively, the screw speed of the extruder can be selected to be from 100 revolutions per minute (rpm) to 300 revolutions per minute (rpm). Preferably the screw speed of the extruder is from 150-250 rpm. Preferably the screw speed of the extruder is from 180-220 rpm. Most preferably the screw speed of the extruder is about 200 rpm.

The melt extrusion process can be carried out at a preferred temperature from 80° C. to 110° C. and at a preferred extruder screw speed from 100 rpm to 300 rpm. More preferably, the melt extrusion temperature can be between 90° C. and 100° C. and the extruder screw speed can be from 150 rpm to 200 rpm. Most preferably, the melt extrusion temperature can be between 93° C. and 98° C. and the extruder screw speed can be about 180-220 rpm. Most preferably the melt extrusion temperature can be about 95° C. and the extruder screw speed can be about 200 rpm.

Preferably the extrusion processing conditions are selected such that the torque is less than or equal to 35 Nm. More preferably the extrusion conditions are selected such that the torque is less than or equal to 25 Nm. Most preferably the extrusion conditions are selected such that the torque is less than or equal to 20 Nm.

Preferably the extrusion conditions are selected such that the torque is more than or equal to 5 Nm. More preferably the extrusion conditions are selected such that the torque is more than or equal to 10 Nm. Most preferably the extrusion conditions are selected such that the torque is more than or equal to 15 Nm.

Preferably the extrusion conditions are selected such that the torque is less than or equal to 35 Nm and more than or equal to 5 Nm. More preferably the extrusion conditions are selected such that the torque is less than or equal to 25 Nm and more than or equal to 10 Nm. Most preferably the extrusion conditions are selected such that the torque is less than or equal to 20 Nm and more than or equal to 15 Nm.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-40% by weight ibuprofen wherein the extrudate can be prepared using a hot melt extrusion process at a temperature between 93° C. and 98° C. and wherein the extruder screw speed can be about 10 rpm.

The extrudate can be comminuted into granules that are suitable for incorporation into an oral dosage form such as oral liquid suspensions, tablets, orodispersible tablets, direct to mouth granules, capsules, lyophilates.

According to a second aspect of the present invention there is provided a taste-masked composition in the form of a solidified melt extrudate comprising an active pharmaceutical ingredient in an amorphous form and pharmaceutically acceptable salts or esters thereof and a combination of a first polymer and a second polymer wherein the composition has a solubility of less than 5% after 5 mins in an aqueous medium at a pH of between 6 and 7.5 and a solubility of more than 5% after 5 mins in an aqueous medium at a pH of between 1 and 5 and wherein the active pharmaceutical agent can be selected from NSAIDs

Preferably the composition can have a solubility of less than 5% after 10 mins in an aqueous medium at a pH of between 6 and 7.5 and a solubility of more than 15% after 10 mins in an aqueous medium at a pH of between 1 and 5. More preferably the composition can have a solubility of less than 5% after 15 mins in an aqueous medium at a pH of between 6 and 7.5 and a solubility of more than 20% after 15 mins in an aqueous medium at a pH of between 1 and 5.

The NSAID can be selected from ibuprofen, flurbiprofen, ketoprofen, diclofenac, naproxen, aspirin, indomethacin, and meloxicam. A preferred NSAID is ibuprofen.

The extrudate can comprise up to 50% by weight of the active pharmaceutical ingredient. The extrudate can comprise up to 45% by weight of the active pharmaceutical ingredient. The extrudate can comprise up to 40% by weight of the active pharmaceutical ingredient. The extrudate can comprise at least 10% by weight of the active pharmaceutical ingredient. The extrudate can comprise at least 15% by weight of the active pharmaceutical ingredient. The extrudate can comprise at least 20% by weight of the active pharmaceutical ingredient.

The extrudate can comprise at least 25% by weight of the active pharmaceutical ingredient. The extrudate can comprise at least 30% by weight of the active pharmaceutical ingredient.

The extrudate can comprise from 10% by weight to 50% by weight of the active pharmaceutical ingredient. The extrudate can comprise from 15% by weight to 45% by weight of the active pharmaceutical ingredient. The extrudate can comprise from 15% by weight to 40% by weight of the active pharmaceutical ingredient. The extrudate can comprise from 20% by weight to 40% by weight of the active pharmaceutical ingredient. The extrudate can comprise from 20% by weight to 30% by weight of the active pharmaceutical ingredient. The extrudate can comprise about 20% by weight of the active pharmaceutical ingredient.

In an alternative embodiment, the extrudate can comprise from 30% by weight to 40% NSAID by weight of the active pharmaceutical ingredient.

Typically, the combination of polymers is miscible with the active pharmaceutical ingredient.

The first polymer of the combination of two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 30° C. and 60° C. The polymer can have a glass transition temperature of between 40° C. and 50° C.

The first polymer is selected such that it provides stability for the amorphous form of the active pharmaceutical ingredient for a period of at least six months.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months.

The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

The first polymer of the combination of two polymers can be present at a level 30-90% by weight of the composition. Preferably the first polymer of the combination of two polymers can be present at a level 40-80% by weight of the composition. More preferably, the first polymer of the combination of two polymers can be present at a level 50-70% by weight of the composition. Most preferably the first polymer of the combination of two polymers can be present at a level of about 60% by weight of the composition.

The second polymer of the combination of two polymers can be selected from polymers having a glass transition temperature of at least 80° C. The polymer can have a glass transition temperature of at least 90° C. The polymer can have a glass transition temperature of less than 120° C. The polymer can have a glass transition temperature of less than 140° C. The polymer can have a glass transition temperature of between 80° C. and 140° C. The polymer can have a glass transition temperature of between 90° C. and 110° C.

Preferably, the solidified melt extrudate comprises ibuprofen in an amorphous form, a combination of two polymers wherein the first polymer can have a glass transition temperature of between 30° C. and 60° C. and the second polymer can have a glass transition temperature of between 80° C. and 140° C. More preferably, the solidified melt extrudate comprises ibuprofen in an amorphous form, a combination of two polymers wherein the first polymer can have a glass transition temperature of between 40° C. and 50° C. and the second polymer can have a glass transition temperature of between 90° C. and 110° C.

The solidified melt extrudate can consist essentially of ibuprofen in an amorphous form, a combination of two polymers wherein the first polymer can have a glass transition temperature of between 30° C. and 60° C. and the second polymer can have a glass transition temperature of between 80° C. and 140° C.

The solidified melt extrudate can consist essentially of ibuprofen in an amorphous form, a combination of two polymers wherein the first polymer can have a glass transition temperature of between 40° C. and 50° C. and the second polymer can have a glass transition temperature of between 90° C. and 110° C.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64). Alternative preferred polymers can be polyvinylpyrrolidone 12 PF or polyvinylpyrrolidone 17 PF.

Alternatively, the second polymer can be selected from a combination of polymers with the proviso that the combination is selected such that the extrudate has a tensile strength of at least 3N/mm². The second polymer can be a combination of polymers selected from polyvinylpyrrolidone, polyvinylpyrrolidone-vinyl acetate co-polymer and polyvinyl caprolactam-polyvinyl acetate-polyethylene glycol graft copolymer.

The second polymer is selected such that the extrudate has a tensile strength of 5-50 N/mm².

The second polymer of the combination of two polymers can be present at a level of 1-30% by weight of the composition. Preferably, the second polymer of the combination of two polymers can be present at a level of 10-25% by weight of the composition. More preferably, the second polymer of the combination of two polymers can be present at a level of 15-25% by weight of the composition. Most preferably, the second polymer of the combination of two polymers can be present at a level of about 20% by weight of the composition.

A preferred combination of the first and second polymers is dimethylaminoethyl methacrylate co-polymer and polyvinylpyrrolidone-vinyl acetate co-polymer (PVPVA64). An alternative preferred combination of the first and second polymers is dimethylaminoethyl methacrylate co-polymer and polyvinylpyrrolidone K12.

The ratio of the first polymer and the second polymer of the combination of two polymers can be selected to be from 10:1 to 1:10. A preferred ratio is from 4:1 to 1:4. A more preferred ratio is from 1:1 to 1:3.

The ratio of the of the active pharmaceutical ingredient, the first polymer and the second polymer can be from 1:1:1 to 1:4:4. Preferably, the ratio of the active pharmaceutical ingredient, the first polymer and the second polymer can be from 1:1.5:1.5 to 1:2.5:2.5. Alternatively, the ratio of the active pharmaceutical ingredient, the first polymer and the second polymer can be from 1:2:1 to 1:4:1.

The active pharmaceutical ingredient is portioned between the each of the first and second polymer of the melt extrudate. Preferably, at least 20% of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate.

Most preferably, from 30% by weight to 35% by weight of the active pharmaceutical ingredient is in the first polymer of the melt extrudate.

The composition can include one or more processing aids or excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid, acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone and croscarmellose sodium.

The surfactants can be selected from polysorbates. A preferred surfactant is polysorbate 80 (Tween 80). Polysorbate 80 can be present as a level of 1-3 w/w %.

The one or more processing aids or excipients such as talc, magnesium silicate and glyceryl monostearate can be added prior to melt extrusion.

The one or more additional processing aids or excipients can be added post-melt extrusion selected from microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 2 to about 4.

Preferably the difference in Hansen solubility parameter between the active pharmaceutical ingredient and one or both of the first polymer and the second polymer is less than 10 MPa^{1/2}. More preferably the difference in Hansen solubility parameter between the active pharmaceutical ingredient and one or both of the combination of the first polymer and the second polymer is less than 7 MPa^{1/2}.

The extrudate is typically prepared using a hot melt extrusion process.

The melt extrusion process is preferably conducted at a temperature of between 80° C. and 110° C. More preferably, the temperature of the extrusion process is between 90° C. and 100° C. Most preferably, the temperature of the extrusion process is between 93° C. and 98° C.

The screw speed of the extruder can be selected from 1 revolution per minute (rpm) to 30 revolutions per minute (rpm). Preferably the screw speed of the extruder is from 5-20 rpm. More preferably the screw speed of the extruder is about 10 rpm.

The melt extrusion process can be carried out at a preferred temperature from 80° C. to 110° C. and at a preferred extruder screw speed from 1rpm to 30 rpm. More preferably, the melt extrusion temperature can be between 90° C. and 100° C. and the extruder screw speed can be from 5 rpm to 20 rpm. Most preferably, the melt extrusion temperature can be between 93° C. and 98° C. and the extruder screw speed can be about 10 rpm.

Alternatively, the screw speed of the extruder can be selected from 100 revolutions per minute (rpm) to 300 revolutions per minute (rpm). Preferably the screw speed of the extruder is from 150-250 rpm. Preferably the screw speed of the extruder is from 180-220 rpm. Most preferably the screw speed of the extruder is about 200 rpm.

The melt extrusion process can be carried out at a preferred temperature from 80° C. to 110° C. and at a preferred extruder screw speed from 100 rpm to 300 rpm. More preferably, the melt extrusion temperature can be between 90° C. and 100° C. and the extruder screw speed can be from 150 rpm to 200 rpm. Most preferably, the melt extrusion temperature can be between 93° C. and 98° C. and the extruder screw speed can be about 180-220 rpm. Most preferably the melt extrusion temperature can be about 95° C. and the extruder screw speed can be about 200 rpm.

Preferably the extrusion processing conditions are selected such that the torque is less than or equal to 35 Nm. More preferably the extrusion conditions are selected such that the torque is less than or equal to 25 Nm. Most preferably the extrusion conditions are selected such that the torque is less than or equal to 20 Nm.

Preferably the extrusion conditions are selected such that the torque is more than or equal to 5 Nm. More preferably the extrusion conditions are selected such that the torque is more than or equal to 10 Nm. Most preferably the extrusion conditions are selected such that the torque is more than or equal to 15 Nm.

Preferably the extrusion conditions are selected such that the torque is less than or equal to 35 Nm and more than or equal to 5 Nm. More preferably the extrusion conditions are selected such that the torque is less than or equal to 25 Nm and more than or equal to 10 Nm. Most preferably the extrusion conditions are selected such that the torque is less than or equal to 20 Nm and more than or equal to 15 Nm.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-40% by weight ibuprofen wherein the extrudate can be prepared using a hot melt extrusion process at a temperature between 93° C. and 98° C. and wherein the extruder screw speed can be about 10 rpm.

The extrudate can be comminuted into granules that are suitable for incorporation into an oral dosage form such as oral liquid suspensions, tablets, orodispersible tablets, direct to mouth granules, capsules, lyophilates.

According to a third aspect of the present invention there is provided the use of a combination of a first polymer and a second polymer as a taste-masking agent for an active pharmaceutical ingredient in an amorphous form and pharmaceutically acceptable salts or esters thereof wherein the active pharmaceutical agent can be selected from NSAIDs wherein the first polymer has a glass transition temperature of between 40° C. and 50° C. and a second polymer has a glass transition temperature of between 90° C. and 110° C. wherein the extrudate can comprise from 30%-45% by weight NSAID.

The NSAID can be selected from ibuprofen, flurbiprofen, ketoprofen, diclofenac, naproxen, aspirin, indomethacin, and meloxicam. A preferred NSAID is ibuprofen.

Typically, the combination of polymers is miscible with the active pharmaceutical ingredient.

Preferably, the extrudate can be prepared using a hot melt extrusion process at a temperature from about 60° C. to about 120° C. More preferably, the extrudate can be prepared using a hot melt extrusion process at a temperature from about 75° C. to about 95° C. Most preferably, the extrudate can be prepared using a hot melt extrusion process at a temperature from about 80° C. to about 90° C.

The solidified melt extrudate can comprise an NSAID in an amorphous form and a combination of a first polymer having a glass transition temperature of between 40° C. and 50° C. and a second polymer having a glass transition temperature of between 90° C. and 110° C. wherein the extrudate can comprise from 30%-45% by weight NSAID wherein the extrudate can be prepared using a holt melt extrusion process at a temperature from about 75° C. to about 95° C.

The first polymer is selected such that it provides stability for the amorphous form of the active pharmaceutical ingredient for a period of at least six months.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the NSAID, the first polymer and the second polymer can be 5:8:1 to 10:15:1. More preferably, the weight ratio of the NSAID, the first polymer and the second polymer can be 7:10:1 to 9:12:1.

Preferably, the solidified melt extrudate comprises 30%-45% by weight of NSAID in an amorphous form and a combination of 45%-65% by weight of the first polymer and 1%-10% by weight of the second polymer. More preferably, the solidified melt extrudate comprises 30%-45% by weight of NSAID in an amorphous form and a combination of 45%-65% by weight of the first polymer and 3%-8% by weight of the second polymer.

The solidified melt extrudate can consist essentially of 30%-45% by weight of NSAID in an amorphous form and a combination of 45%-65% by weight of the first polymer and 1%-10% by weight of the second polymer.

The solidified melt extrudate can consist essentially of 30%-45% by weight of NSAID in an amorphous form and a combination of 45%-65% by weight of the first polymer and 3%-8% by weight of the second polymer.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C. More preferably, the solidified melt extrudate comprises 30%-45% by weight of NSAID in an amorphous form and a combination of 45%-65% by weight of the first polymer and 3%-8% by weight of the second polymer wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein the weight ratio of the NSAID, the first polymer and the second polymer is from about 5:8:1 to about 10:15:1.

More preferably, the solidified melt extrudate comprises 30%-45% by weight of an NSAID in an amorphous form and a combination of 45%-65% by weight of the first polymer and 4%-7% by weight of the second polymer wherein the extrudate can be prepared using a hot melt extrusion process at a temperature from about 75° C. to about 95° C. and wherein the weight ratio of the NSAID, the first polymer and the second polymer is from about 7:10:1 to about 9:12:1.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons.

More preferably, the solidified melt extrudate comprises 30%-45% by weight of an NSAID in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

Preferably, when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64). Alternative preferred polymers can be polyvinylpyrrolidone 12 PF or polyvinylpyrrolidone 17 PF.

The second polymer is selected such that the extrudate has a tensile strength of 5-50 N/mm².

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

The composition can include one or more processing aids or excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid, acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone and croscarmellose sodium.

The surfactants can be selected from polysorbates. A preferred surfactant is polysorbate 80 (Tween 80). Polysorbate 80 can be present as a level of 1-3 w/w %.

The one or more excipients or processing aids such as silicon dioxide, talc, magnesium silicate and glyceryl monostearate can be added prior to melt extrusion.

The one or more additional excipients processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises an NSAID in an amorphous form and a combination of dimethylaminoethyl methacrylate co-polymer and polyvinylpyrrolidone-vinyl acetate co-polymer (PVPVA64) wherein the extrudate can comprise from 30%-45% by weight NSAID wherein the extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the NSAID, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64). More preferably, the solidified melt extrudate comprises 30%-45% by weight of NSAID in an amorphous form and a combination of 45%-65% by weight of dimethylaminoethyl methacrylate co-polymer and 3%-8% by weight of polyvinylpyrrolidone-vinyl acetate co-polymer (PVPVA64).

The solidified melt extrudate can consist essentially of 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64).

The solidified melt extrudate consist essentially of 30%-45% by weight of NSAID in an amorphous form and a combination of 45%-65% by weight of dimethylaminoethyl methacrylate co-polymer and 3%-8% by weight of polyvinylpyrrolidone-vinyl acetate co-polymer (PVPVA64).

Preferably, the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C. More preferably, the solidified melt extrudate comprises 30%-45% by weight of NSAID in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the NSAID, the dimethylaminoethyl methacrylate co-polymer and the polyvinylpyrrolidone-vinyl acetate co-polymer (PVPVA64) is from about 5:8:1 to about 10:15:1.

More preferably, the solidified melt extrudate comprises 30%-45% by weight of an NSAID in an amorphous form and a combination of 45%-65% by weight of the first polymer and 4%-7% by weight of the second polymer wherein the extrudate can be prepared using a hot melt extrusion process at a temperature from about 75° C. to about 95° C. and wherein the weight ratio of the NSAID, the first polymer and the second polymer is from about 7:10:1 to about 9:12:1.

Preferably, the solidified melt extrudate comprises ibuprofen in an amorphous form and a combination of a first polymer having a glass transition temperature of between 40° C. and 50° C. and a second polymer having a glass transition temperature of between 90° C. and 110° C. wherein the extrudate can comprise from 30%-45% by weight of ibuprofen. The extrudate can be prepared using a hot melt extrusion process at a temperature from about 75° C. to about 95° C.

Typically, the combination of polymers is miscible with the active pharmaceutical ingredient.

The first polymer is selected such that it provides stability for the amorphous form of the active pharmaceutical ingredient for a period of at least six months.

Preferably, when the second polymer of the combination of two polymers is selected to be a homopolymer it has an average molecular weight of 1,000 to 5,000 Daltons and when the second polymer of the combination of two polymers is selected to be a co-polymer it has an average molecular weight of from 45,000 to 70,000 Daltons.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the ibuprofen, the first polymer and the second polymer can be 5:8:1 to 10:15:1. More preferably, the weight ratio of the ibuprofen, the first polymer and the second polymer can be 7:10:1 to 9:12:1.

Preferably, the solidified melt extrudate comprises 30%-45% by weight of ibuprofen in an amorphous form and a combination of 45%-65% by weight of the first polymer and 1%-10% by weight of the second polymer. More preferably, the solidified melt extrudate comprises 30%-45% by weight of ibuprofen in an amorphous form and a combination of 45%-65% by weight of the first polymer and 3%-8% by weight of the second polymer.

The solidified melt extrudate can consist essentially of 30%-45% by weight of ibuprofen in an amorphous form and a combination of 45%-65% by weight of the first polymer and 1%-10% by weight of the second polymer.

The solidified melt extrudate can consist essentially of 30%-45% by weight of ibuprofen in an amorphous form and a combination of 45%-65% by weight of the first polymer and 3%-8% by weight of the second polymer.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. More preferably, the solidified melt extrudate comprises 30%-45% by weight of ibuprofen in an amorphous form and a combination of 45%-65% by weight of the first polymer and 3%-8% by weight of the second polymer wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein the weight ratio of the ibuprofen, the first polymer and the second polymer is from about 5:8:1 to about 10:15:1.

More preferably, the solidified melt extrudate comprises 30%-45% by weight of an NSAID in an amorphous form and a combination of 45%-65% by weight of the first polymer and 4%-7% by weight of the second polymer wherein the extrudate can be prepared using a hot melt extrusion process at a temperature from about 75° C. to about 95° C. and wherein the weight ratio of the NSAID, the first polymer and the second polymer is from about 7:10:1 to about 9:12:1.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons. More preferably, the solidified melt extrudate comprises 30%-45% by weight of ibuprofen in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64). Alternative preferred polymers can be polyvinylpyrrolidone 12 PF or polyvinylpyrrolidone 17 PF.

The second polymer is selected such that the extrudate has a tensile strength of 5-50 N/mm².

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

The composition can include one or more processing aids or excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid, acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone and croscarmellose sodium.

The surfactants can be selected from polysorbates. A preferred surfactant is polysorbate 80 (Tween 80). Polysorbate 80 can be present as a level of 1-3 w/w %.

The one or more processing aids or excipients such as silicon dioxide, talc, magnesium silicate and glyceryl monostearate can be added prior to melt extrusion.

The one or more additional processing aids or excipients can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-45% by weight of ibuprofen wherein the extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the NSAID, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64). More preferably, the solidified melt extrudate comprises 30%-45% by weight of ibuprofen in an amorphous form and a combination of 45%-65% by weight of dimethylaminoethyl methacrylate co-polymer and 3%-8% by weight the polyvinylpyrrolidone-vinyl acetate co-polymer (PVPVA64).

The solidified melt extrudate can consist essentially of 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64).

The solidified melt extrudate can consist essentially of 30%-45% by weight of ibuprofen in an amorphous form and a combination of 45%-65% by weight of dimethylaminoethyl methacrylate co-polymer and 3%-8% by weight the polyvinylpyrrolidone-vinyl acetate co-polymer (PVPVA64).

Preferably, the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64) wherein the extrudate can be prepared using a hot melt extrusion process at a temperature from about 75° C. to about 95° C. More preferably, the solidified melt extrudate comprises 30%-45% by weight of ibuprofen in an amorphous form and a combination of 45%-65% by weight of dimethylaminoethyl methacrylate co-polymer and 3%-8% by weight the polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the ibuprofen, the dimethylaminoethyl methacrylate co-polymer and the polyvinylpyrrolidone-vinyl acetate co-polymer (PVPVA64) is from about 5:8:1 to about 10:15:1.

More preferably, the solidified melt extrudate comprises 30%-45% by weight of an NSAID in an amorphous form and a combination of 45%-65% by weight of the first polymer and 4%-7% by weight of the second polymer wherein the extrudate can be prepared using a hot melt extrusion process at a temperature from about 75° C. to about 95° C. and wherein the weight ratio of the NSAID, the first polymer and the second polymer is from about 7:10:1 to about 9:12:1.

According to a fourth aspect of the present invention there is provided a taste-masked composition in the form of a solidified melt extrudate comprising an active pharmaceutical ingredient in an amorphous form and pharmaceutically acceptable salts or esters thereof and a combination of a first polymer and a second polymer wherein the composition has a solubility of less than 5% after 5 mins in an aqueous medium at a pH of between 6 and 7.5 and a solubility of more than 5% after 5 mins in an aqueous medium at a pH of between 1 and 5 and wherein the active pharmaceutical agent can be selected from NSAIDs, the first polymer has a glass transition temperature of between 40° C. and 50° C. and a second polymer has a glass transition temperature of between 90° C. and 110° C. wherein the extrudate can 5 comprise from 30%-45% by weight NSAID.

Preferably the composition can have a solubility of less than 5% after 10 mins in an aqueous medium at a pH of between 6 and 7.5 and a solubility of more than 15% after 10 mins in an aqueous medium at a pH of between 1 and 5. More preferably the composition can have a solubility of less than 5% after 15 mins in an aqueous medium at a pH of between 6 and 7.5 and a solubility of more than 20% after 15 mins in an aqueous medium at a pH of between 1 and 5.

The NSAID can be selected from ibuprofen, flurbiprofen, ketoprofen, diclofenac, naproxen, aspirin, indomethacin, and meloxicam. A preferred NSAID is ibuprofen.

Typically, the combination of polymers is miscible with the active pharmaceutical ingredient.

Preferably, the extrudate can be prepared using a hot melt extrusion process at a temperature from about 60° C. to about 120° C. More preferably, the extrudate can be prepared using a hot melt extrusion process at a temperature from about 75° C. to about 95° C. Most preferably, the extrudate can be prepared using a hot melt extrusion process at a temperature from about 80° C. to about 90° C.

The solidified melt extrudate can comprise an NSAID in an amorphous form and a combination of a first polymer having a glass transition temperature of between 40° C. and 50° C. and a second polymer having a glass transition temperature of between 90° C. and 110° C. wherein the extrudate can comprise from 30%-45% by weight NSAID wherein the extrudate can be prepared using a holt melt extrusion process at a temperature from about 75° C. to about 95° C.

The first polymer is selected such that it provides stability for the amorphous form of the active pharmaceutical ingredient for a period of at least six months.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the NSAID, the first polymer and the second polymer can be 5:8:1 to 10:15:1. More preferably, the weight ratio of the NSAID, the first polymer and the second polymer can be 7:10:1 to 9:12:1.

Preferably, the solidified melt extrudate comprises 30%-45% by weight of NSAID in an amorphous form and a combination of 45%-65% by weight of the first polymer and 1%-10% by weight of the second polymer.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein the weight ratio of the NSAID, the first polymer and the second polymer is from about 5:8:1 to about 10:15:1.

More preferably, the solidified melt extrudate comprises 30%-45% by weight of an NSAID in an amorphous form and a combination of 45%-65% by weight of the first polymer and 4%-7% by weight of the second polymer wherein the extrudate can be prepared using a hot melt extrusion process at a temperature from about 75° C. to about 95° C. and wherein the weight ratio of the NSAID, the first polymer and the second polymer is from about 7:10:1 to about 9:12:1.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons. More preferably, the solidified melt extrudate comprises 30%-45% by weight of an NSAID in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

Preferably, when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64). Alternative preferred polymers can be polyvinylpyrrolidone 12 PF or polyvinylpyrrolidone 17 PF.

The second polymer is selected such that the extrudate has a tensile strength of 5-50 N/mm².

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

The composition can include one or more processing aids or excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid, acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone and croscarmellose sodium.

The surfactants can be selected from polysorbates. A preferred surfactant is polysorbate 80 (Tween 80). Polysorbate 80 can be present as a level of 1-3 w/w %.

The one or more excipients or processing aids such as silicon dioxide, talc, magnesium silicate and glyceryl monostearate can be added prior to melt extrusion.

The one or more additional excipients processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises an NSAID in an amorphous form and a combination of dimethylaminoethyl methacrylate co-polymer and polyvinylpyrrolidone-vinyl acetate co-polymer (PVPVA64) wherein the extrudate can comprise from 30%-45% by weight NSAID wherein the extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the NSAID, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64). More preferably, the solidified melt extrudate comprises 30%-45% by weight of NSAID in an amorphous form and a combination of 45%-65% by weight of dimethylaminoethyl methacrylate co-polymer and 3%-8% by weight of polyvinylpyrrolidone-vinyl acetate co-polymer (PVPVA64).

The solidified melt extrudate can consist essentially of 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64).

The solidified melt extrudate can consist essentially of 30%-45% by weight of NSAID in an amorphous form and a combination of 45%-65% by weight of dimethylaminoethyl methacrylate co-polymer and 3%-8% by weight of polyvinylpyrrolidone-vinyl acetate co-polymer (PVPVA64).

Preferably, the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C. More preferably, the solidified melt extrudate comprises 30%-45% by weight of NSAID in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the NSAID, the dimethylaminoethyl methacrylate co-polymer and the polyvinylpyrrolidone-vinyl acetate co-polymer (PVPVA64) is from about 5:8:1 to about 10:15:1.

More preferably, the solidified melt extrudate comprises 30%-45% by weight of an NSAID in an amorphous form and a combination of 45%-65% by weight of dimethylaminoethyl methacrylate co-polymer and 4%-7% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the NSAID, the dimethylaminoethyl methacrylate co-polymer and polyvinylpyrrolidone-vinyl acetate co-polymer (PVPVA64) is from about 7:10:1 to about 9:12:1.

Preferably, the solidified melt extrudate comprises ibuprofen in an amorphous form and a combination of a first polymer having a glass transition temperature of between 40° C. and 50° C. and a second polymer having a glass transition temperature of between 90° C. and 110° C. wherein the extrudate can comprise from 30%-45% by weight of ibuprofen. The extrudate can be prepared using a hot melt extrusion process at a temperature from about 75° C. to about 95° C.

Typically, the combination of polymers is miscible with the active pharmaceutical ingredient.

The first polymer is selected such that it provides stability for the amorphous form of the active pharmaceutical ingredient for a period of at least six months.

Preferably, when the second polymer of the combination of two polymers is selected to be a homopolymer it has an average molecular weight of 1,000 to 5,000 Daltons and when the second polymer of the combination of two polymers is selected to be a co-polymer it has an average molecular weight of from 45,000 to 70,000 Daltons.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the ibuprofen, the first polymer and the second polymer can be 5:8:1 to 10:15:1. More preferably, the weight ratio of the ibuprofen, the first polymer and the second polymer can be 7:10:1 to 9:12:1.

Preferably, the solidified melt extrudate comprises 30%-45% by weight of ibuprofen in an amorphous form and a combination of 45%-65% by weight of the first polymer and 1%-10% by weight of the second polymer. More preferably, the solidified melt extrudate comprises 30%-45% by weight of ibuprofen in an amorphous form and a combination of 45%-65% by weight of the first polymer and 3%-8% by weight of the second polymer. The solidified melt extrudate can consist essentially of 30%-45% by weight of ibuprofen in an amorphous form and a combination of 45%-65% by weight of the first polymer and 1%-10% by weight of the second polymer.

The solidified melt extrudate can consist essentially of 30%-45% by weight of ibuprofen in an amorphous form and a combination of 45%-65% by weight of the first polymer and 3%-8% by weight of the second polymer.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. More preferably, the solidified melt extrudate comprises 30%-45% by weight of ibuprofen in an amorphous form and a combination of 45%-65% by weight of the first polymer and 3%-8% by weight of the second polymer wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein the weight ratio of the ibuprofen, the first polymer and the second polymer is from about 5:8:1 to about 10:15:1.

More preferably, the solidified melt extrudate comprises 30%-45% by weight of an ibuprofen in an amorphous form and a combination of 45%-65% by weight of the first polymer and 4%-7% by weight of the second polymer wherein the extrudate can be prepared using a hot melt extrusion process at a temperature from about 75° C. to about 95° C. and wherein the weight ratio of the ibuprofen, the first polymer and the second polymer is from about 7:10:1 to about 9:12:1.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons. More preferably, the solidified melt extrudate comprises 30%-45% by weight of ibuprofen in an amorphous form and a combination of 45%-65% by weight of the first polymer and 3%-8% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64). Alternative preferred polymers can be polyvinylpyrrolidone 12 PF or polyvinylpyrrolidone 17 PF.

The second polymer is selected such that the extrudate has a tensile strength of 5-50 N/mm².

The ibuprofen is portioned between the each of the first and second polymer of the melt extrudate. Preferably, at least 20% of the of the ibuprofen is in the first polymer. More preferably at least 25% is in the first polymer. Most preferably at least 30% is in the first polymer. Preferably, less than or equal to 50% of the ibuprofen is in the first polymer. More preferably less than or equal to 40% is in the first polymer. Most preferably less than or equal to 35% is in the first polymer.

Preferably, from 20% by weight to 50% by weight of the ibuprofen is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

The composition can include one or more processing aids or excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid, acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone and croscarmellose sodium.

The surfactants can be selected from polysorbates. A preferred surfactant is polysorbate 80 (Tween 80). Polysorbate 80 can be present as a level of 1-3 w/w %.

The one or more processing aids or excipients such as silicon dioxide, talc, magnesium silicate and glyceryl monostearate can be added prior to melt extrusion.

The one or more additional processing aids or excipients can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-45% by weight of ibuprofen wherein the extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the ibuprofen, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64). More preferably, the solidified melt extrudate comprises 30%-45% by weight of ibuprofen in an amorphous form and a combination of 45%-65% by weight of dimethylaminoethyl methacrylate co-polymer and 3%-8% by weight the polyvinylpyrrolidone-vinyl acetate co-polymer (PVPVA64).

The solidified melt extrudate can consist essentially of 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64).

The solidified melt extrudate can consist essentially of 30%-45% by weight of ibuprofen in an amorphous form and a combination of 45%-65% by weight of dimethylaminoethyl methacrylate co-polymer and 3%-8% by weight the polyvinylpyrrolidone-vinyl acetate co-polymer (PVPVA64).

Preferably, the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64) wherein the extrudate can be prepared using a hot melt extrusion process at a temperature from about 75° C. to about 95° C. More preferably, the solidified melt extrudate comprises 30%-45% by weight of ibuprofen in an amorphous form and a combination of 45%-65% by weight of dimethylaminoethyl methacrylate co-polymer and 3%-8% by weight the polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the ibuprofen, the dimethylaminoethyl methacrylate co-polymer and the polyvinylpyrrolidone-vinyl acetate co-polymer (PVPVA64) is from about 5:8:1 to about 10:15:1.

More preferably, the solidified melt extrudate comprises 30%-45% by weight of an ibuprofen in an amorphous form and a combination of 45%-65% by weight of the first polymer and 4%-7% by weight of the second polymer wherein the extrudate can be prepared using a hot melt extrusion process at a temperature from about 75° C. to about 95° C. and wherein the weight ratio of the ibuprofen, the first polymer and the second polymer is from about 7:10:1 to about 9:12:1.

According to a fifth aspect of the present invention there is provided a method of taste-masking an active pharmaceutical ingredient in an amorphous form by using a combination of a first polymer and a second polymer as described in any of the previous aspects of the present invention wherein the active pharmaceutical agent can be selected from NSAIDs wherein the active pharmaceutical ingredient and the combination of the first polymer and the second polymer are in the form of a solidified melt extrudate.

The solidified melt extrudates of the present invention are prepared using a single melt extrusion process, i.e. the active pharmaceutical ingredient and the combination of the two polymers are simultaneously fed and extruded in a single hot melt extrusion process.

For the avoidance of doubt the term “amorphous” has the meaning that the ibuprofen has no defined crystalline structure characteristic of conventional ibuprofen.

For the avoidance of doubt the active pharmaceutical ingredient can be in either a crystalline form or an amorphous form prior to the melt extrusion process.

For the avoidance of doubt the term “torque” refers to the force needed to cause the screw in the extruder to turn.

The Hansen solubility parameter is a numerical value used to indicate the relative solvency of a particular material. It is typically used to determine if a particular material will dissolve in another material, and is well-known to the person skilled in the art.

In the context of the present invention the terms “granule” and “granules” as used herein refer to discrete particle or particles and includes pellets, powders or spheres.

Embodiments of the present invention will now be described, by way of example only, with reference to the accompanying Figures in which:

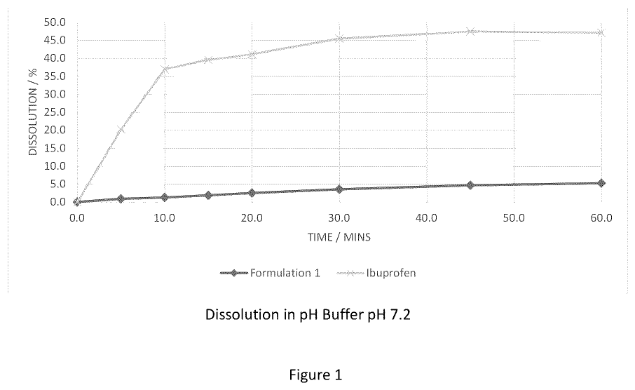

FIG. 1 illustrates the dissolution profile for an example of the present invention and an existing ibuprofen composition at a pH of 7.2; and

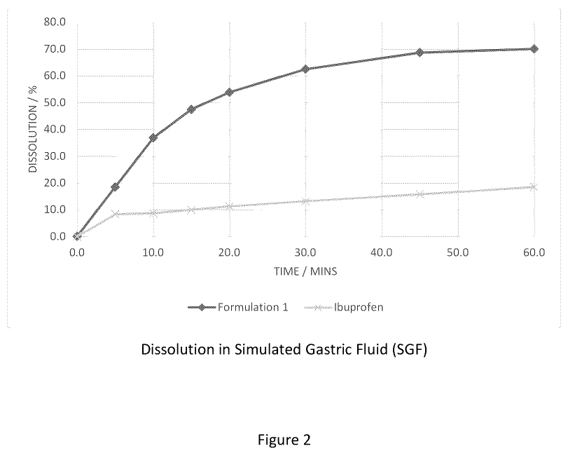

FIG. 2 illustrates the dissolution profile for an example of the present invention and an existing ibuprofen composition in simulated gastric fluid (pH 1.2).

### Materials and Methods

Crystalline ibuprofen (MW: 206.2 g/mol) obtained from Shasun (India), Dimethylaminoethyl Methacrylate Copolymer Eudragit® E PO (MW: 147,000 g/mol) obtained from Evonik (Germany), Polyvinylpyrrolidone Kollidon® K12 (MW: 2000-3000 g/mol), Polyvinylpyrrolidone-vinyl acetate copolymers Kollidon® VA64 (MW: 15,000-20,000 g/mol) and polyvinyl caprolactam-polyvinyl acetate-polyethylene glycol graft copolymer Soluplus® (MW: 90,000-140,000 g/mol) obtained from BASF (Germany). All other materials were of analytical grade and used without further treatment.

Ibuprofen, Eudragit (EPO) and either PVPK12 or PVPVA64 were extruded using a Rondol 10 mm co-rotating fully intermeshing twin screw extruder. Barrel temperature was kept at 95° C. during the extrusion process and three screw speeds were used (namely 5, 10, 20 rpm). Full conveying screw geometry was used in all extrusion experiments. Extrudates were stored in glass vials and used for further characterisation. Extrudates based on individual polymers were manufactured at 10 rpm screw speed and 95° C. barrel temperature and used as a standard for comparison.

Extrudates having similar properties could also be manufactured using a Leistritz Nano 16 (Somerville, NJ, USA), which is a co-rotating twin screw extruder (screw diameter 16 mm) with three heating zones and a die zone. Each formulation ingredient was sieved and then blended. The formulation pre-mix was transferred to a feeder. The pre-mix was fed into the extruder at a rate of 7 g/min. The feeding zone has a water jacket cooling system kept at 5° C. The extruder barrel was operated at 200 rpm and the temperature of the die was 95° C. The formulation was heated in the extruder barrel and the molten extrudate was passed on to a 2 m conveyor belt cooled with compressed air prior to milling. The samples could be milled at room temperature if required.

Example formulations are shown below in Table 1:

| | Formulation 1 | Formulation 2 | Formulation 3 |
|---|---|---|---|
| Material | (% w/w) | (% w/w) | (% w/w) |
| Ibuprofen | 20 | 40 | 40 |
| Eudragit | 60 | 55 | 40 |
| Kollidon VA64 | 20 | 5 | |
| Kollidon K12 | — | — | 20 |
| TOTAL | 100 | 100 | 100 |

The dissolution studies were performed as follows. USP Type II apparatus as defined by the United States Pharmacopoeia (USP) was used. The procedure followed was that set out in USP 711.

Two dissolution baths were used-one bath was provided with a 900 ml of a phosphate buffer at a pH of 7.2 and a temperature of 37° C., and the other bath was provided with 900 ml of simulated gastric fluid (pH 1.2) and a temperature of 37° C. The paddles in each bath were rotated at 50 RPM throughout.

Simulated gastric fluid was prepared as per EP 9.05.17.1. which involved dissolving 2 g sodium chloride in 80 ml hydrochloric acid and 920 ml deionised water.

Each dissolution bath had an automated in-line UV sampler which allowed samples to be taken at set time points. The samples were then run through a UV detector to calculate the percentage of ibuprofen dissolved. Samples were taken at 5, 10, 15, 20, 30, 45 and 60 minutes and the UV was set to take readings at 276 nm.

An advantage of the present invention is that there is provided a composition with excellent taste-masking properties as a result of the solubility of active pharmaceutical ingredient being minimised at oral pH. The composition can be incorporated into an oral dosage form without the need for the inclusion an additional agent to mask the unpleasant or bitter taste of the active pharmaceutical ingredient. The formulation of the present invention also has an improved dissolution profile.

The present invention is not intended to be limited to the exemplary embodiments described herein. Further modifications can be made without departing from the scope of the invention described herein.

1. The use of a combination of a first polymer and a second polymer as a taste-masking agent for an active pharmaceutical ingredient in an amorphous form and pharmaceutically acceptable salts or esters thereof wherein the active pharmaceutical agent is selected from NSAIDs wherein the active pharmaceutical ingredient and the combination of the first polymer and the second polymer are in the form of a solidified melt extrudate.

2. A taste-masked composition comprising an active pharmaceutical ingredient and a combination of a first polymer and a second polymer wherein the composition has a solubility of less than 5% after 5 mins at a pH of between 6 and 7.5 and a solubility of more than 5% after 5 mins at a pH of between 1 and 5 and wherein the active pharmaceutical ingredient and the combination of the first polymer and the second polymer are in the form of a solidified melt extrudate.

3. The use as claimed in claim 1 or the composition as claimed in claim 2 wherein the NSAID is selected from ibuprofen, flurbiprofen, ketoprofen, diclofenac, naproxen, aspirin, indomethacin, and meloxicam.

4. The use or composition as claim in claim 3 wherein the NSAID is ibuprofen.

5. The use or composition as claimed in any of the preceding claims wherein the extrudate comprises from 10% by weight to 50% by weight of the active pharmaceutical ingredient.

6. The use or composition as claimed in claim 5 wherein the extrudate comprises from 30% by weight to 40% NSAID by weight of the active pharmaceutical ingredient.

7. The use or composition as claimed in any of the preceding claims wherein the first polymer of the combination of at least two polymers has a glass transition temperature of between 40° C. and 50° C.

8. The use or composition as claimed in any of the preceding claims wherein the first polymer of the combination of two polymers is selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, poly(vinylmethyl ether/maleic anhydride), crospovidone, croscarmellose sodium, dimethylaminoethyl methacrylate co-polymer.

9. The use or composition as claimed in claim 8 wherein the first polymer is dimethylaminoethyl methacrylate co-polymer.

10. The use or composition as claimed in any of the preceding claims wherein the first polymer of the combination of two polymers is present at a level 30-90% by weight of the composition.

11. The use or composition as claimed in any of the preceding claims wherein the first polymer of the combination of two polymers is present at a level 50-70% by weight of the composition.

12. The use or composition as claimed in claim 11 wherein the first polymer has a glass transition temperature of between 90° C. and 110° C.

13. The use or composition as claimed in any of the preceding claims wherein the second polymer of the combination of two polymers is selected from polyvinylpyrrolidone, polyvinylpyrrolidone-vinyl acetate co-polymer and polyvinyl caprolactam-polyvinyl acetate-polyethylene glycol graft copolymer.

14. The use or composition as claimed in claim 13 wherein the second polymer is polyvinylpyrrolidone K12 or polyvinylpyrrolidone-vinyl acetate co-polymer (PVPVA64).

15. The use or composition as claimed in any of the preceding claims wherein the second polymer of the combination of two polymers is present in a level of 1-30% by weight of the composition.

16. The use or composition as claimed in claim 15 wherein the second polymer of the combination of two polymers is present in a level of 15-25% by weight of the composition.

17. The use or composition as claimed in any of the preceding claims wherein the combination of the first and second polymers is dimethylaminoethyl methacrylate co-polymer and polyvinylpyrrolidone-vinyl acetate co-polymer (PVPVA64).

18. The use or composition as claimed in any of the preceding claims wherein the ratio of the first polymer and the second polymer of the combination of two polymers is from 10:1 to 1:10.

19. The use or composition as claimed in in claim 18 wherein the ratio of the first polymer and the second polymer of the combination of two polymers is from 1:1 to 1:3.

20. The use or composition as claimed in any of the preceding claims wherein the ratio of the of the active pharmaceutical ingredient, the first polymer and the second polymer is from 1:1:1 to 1:4:4.

21. The use or composition as claimed in claim 20 wherein the ratio of the active pharmaceutical ingredient, the first polymer and the second polymer is from 1:2:1 to 1:4:1.

22. The use or composition as claimed in any of the preceding claims wherein the active pharmaceutical ingredient is portioned between the each of the first and second polymer of the melt extrudate wherein from 20% by weight to 50% by weight of the active pharmaceutical ingredient is in the first polymer of the melt extrudate.

23. The use or composition as claimed in claim 22 wherein from 30% by weight to 35% by weight of the active pharmaceutical ingredient is in the first polymer of the melt extrudate.

24. The use or composition as claimed in any of the preceding claims wherein the combination of polymers is soluble at a pH of about 1 to about 5.

25. The use or composition as claimed in any of the preceding claims wherein the combination of polymers is soluble at a pH of about 2 to about 4.

26. The use or composition as claimed in any of the preceding claims wherein the difference in Hansen solubility parameter between the active pharmaceutical ingredient and the combination of the first polymer and the second polymer is less than 10 MPa^{1/2}.

27. The use or composition as claimed in claim 26 wherein the difference in Hansen solubility parameter between the active pharmaceutical ingredient and the combination of the first polymer and the second polymer is less than 7 MPa^{1/2}.

28. The use or composition as claimed in any of the preceding claims wherein the extrudate is prepared using a hot melt extrusion process.

29. The use or composition as claimed in claim 28 wherein the melt extrusion process is conducted at a temperature of between 80° C. and 110° C.

30. The use or composition as claimed in in claim 28 or claim 29 wherein the temperature of the extrusion process is between 93° C. and 98° C.

31. The use or composition as claimed in any of claims 28-30 wherein the screw speed of the extruder is from 1 revolution per minute (rpm) to 30 revolutions per minute (rpm).

32. The use or composition as claimed in in claim 31 wherein the screw speed of the extruder is about 10 rpm.

33. The use or composition as claimed in any of claims 28-32 wherein the melt extrusion process is carried out at a temperature from 80° C. to 110° C. and at an extruder screw speed from 1 rpm to 30 rpm.

34. The use or composition as claimed in any of claims 28-33 wherein the melt extrusion temperature is between 93° C. and 98° C. and the extruder screw speed is about 10 rpm.

35. The use or composition as claimed in any of claims 28-30 wherein the screw speed of the extruder is from 100 revolutions per minute (rpm) to 300 revolutions per minute (rpm).

36. The use or composition as claimed in claim 35 wherein the screw speed of the extruder is from 180-220 rpm.

37. The use or composition as claimed in claim 36 or claim 37 wherein the melt extrusion process is carried out at a temperature from 80° C. to 110° C. and at an extruder screw speed from 100 rpm to 300 rpm.

38. The use or composition as claimed in in claim 37 wherein the melt extrusion temperature is between 93° C. and 98° C. and the extruder screw speed is about 180-220 rpm.

39. The use or composition as claimed in any of claims 28-38 wherein the extrusion processing conditions are selected such that the torque is less than or equal to 35 Nm.

40. The use or composition as claimed in claim 39 wherein the extrusion conditions are selected such that the torque is less than or equal to 20 Nm.

41. The use or composition as claimed in claim 39 or claim 40 wherein the extrusion conditions are selected such that the torque is more than or equal to 5 Nm.

42. The use or composition as claimed in any of claims 39-41 wherein the extrusion conditions are selected such that the torque is more than or equal to 15 Nm.

43. The use or composition as claimed in claim 39 wherein the extrusion conditions are selected such that the torque is less than or equal to 35 Nm and more than or equal to 5 Nm.

44. The use or composition as claimed in claim 39 wherein the extrusion conditions are selected such that the torque is less than or equal to 20 Nm and more than or equal to 15 Nm.

45. The use or composition as claimed in any of claims 1-34 wherein the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate comprises from 30%-40% by weight ibuprofen wherein the extrudate is prepared using a hot melt extrusion process at a temperature between 93° C. and 98° C. and wherein the extruder screw speed is about 10 rpm.

46. The use of a combination of a first polymer and a second polymer as a taste-masking agent for an active pharmaceutical ingredient in an amorphous form and pharmaceutically acceptable salts or esters thereof wherein the active pharmaceutical agent is selected from NSAIDs wherein the first polymer has a glass transition temperature of between 40° C. and 50° C. and a second polymer has a glass transition temperature of between 90° C. and 110° C. wherein the extrudate comprises from 30%-45% by weight NSAID.

47. A taste-masked composition in the form of a solidified melt extrudate comprising an active pharmaceutical ingredient in an amorphous form and pharmaceutically acceptable salts or esters thereof and a combination of a first polymer and a second polymer wherein the composition has a solubility of less than 5% after 5 mins in an aqueous medium at a pH of between 6 and 7.5 and a solubility of more than 5% after 5 mins in an aqueous medium at a pH of between 1 and 5 and wherein the active pharmaceutical agent is selected from NSAIDs, the first polymer has a glass transition temperature of between 40° C. and 50° C. and a second polymer has a glass transition temperature of between 90° C. and 110° C. wherein the extrudate comprises from 30%-45% by weight NSAID.

48. A composition as claimed in claim 47 wherein the composition has a solubility of less than 5% after 15 mins in an aqueous medium at a pH of between 6 and 7.5 and a solubility of more than 20% after 15 mins in an aqueous medium at a pH of between 1 and 5.

49. The use as claimed in claim 46 or the composition as claimed in claim 47 or claim 48 wherein the NSAID is selected from ibuprofen, flurbiprofen, ketoprofen, diclofenac, naproxen, aspirin, indomethacin, and meloxicam.

50. The use or composition as claimed in claim 49 wherein the NSAID is ibuprofen.

51. The use or composition as claimed in any of claims 46-50 wherein the extrudate is prepared using a hot melt extrusion process at a temperature from about 60° C. to about 120° C.

52. The use or composition as claimed in any of claims 46-51 wherein the solidified melt extrudate comprises an NSAID in an amorphous form and a combination of a first polymer having a glass transition temperature of between 40° C. and 50° C. and a second polymer having a glass transition temperature of between 90° C. and 110° C. wherein the extrudate comprises from 30%-45% by weight NSAID wherein the extrudate is prepared using a holt melt extrusion process at a temperature from about 75° C. to about 95° C.

53. The use or composition as claimed in any of claims 46-52 wherein the weight ratio of the of the first polymer and the second polymer is from 5:1 to 15:1.

54. The use or composition as claimed in any of claims 46-52 wherein the weight ratio of the NSAID, the first polymer and the second polymer is from 5:8:1 to 10:15:1.

55. The use or composition as claimed in any of claims 46-52 wherein the solidified melt extrudate comprises 30%-45% by weight of NSAID in an amorphous form and a combination of 45%-65% by weight of the first polymer and 1%-10% by weight of the second polymer.

56. The use or composition as claimed in claim 55 wherein the solidified melt extrudate comprises 30%-45% by weight of an NSAID in an amorphous form and a combination of 45%-65% by weight of the first polymer and 1%-10% by weight of the second polymer wherein the extrudate is prepared using a hot melt extrusion process at a temperature from about 75° C. to about 95° C.

57. The use or composition as claimed in claim 56 wherein the solidified melt extrudate comprises 30%-45% by weight of an NSAID in an amorphous form and a combination of 45%-65% by weight of the first polymer and 3%-8% by weight of the second polymer wherein the extrudate is prepared using a hot melt extrusion process at a temperature from about 75° C. to about 95° C. and wherein the weight ratio of the NSAID, the first polymer and the second polymer is from about 5:8:1 to about 10:15:1.

58. The use or composition as claimed in any of claims 46-52 wherein the solidified melt extrudate comprises 30%-45% by weight of an NSAID in an amorphous form and a combination of 45%-65% by weight of the first polymer and 1%-10% by weight of the second polymer wherein the extrudate is prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons.

59. The use or composition as claimed in any of claims 46-58 wherein the first polymer of the combination of two polymers is selected from polymethacrylates including but not limited to amino methacrylate copolymer, amino-methyl methacrylate, methacrylic acid methyl methacrylate, dimethylaminoethyl methacrylate co-polymer.

60. The use or composition as claimed in any of claims 46-59 wherein when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

61. The use or composition as claimed in any of claims 46-62 wherein the second polymer of the combination of two polymers is selected from polyvinylpyrrolidone, polyvinylpyrrolidone-vinyl acetate co-polymer and polyvinyl caprolactam-polyvinyl acetate-polyethylene glycol graft copolymer.

62. The use or composition as claimed in claim 61 wherein the second polymer is polyvinylpyrrolidone K12 or polyvinylpyrrolidone-vinyl acetate co-polymer (PVPVA64).

63. The use or composition as claimed in any of claims 46-62 wherein the combination of polymers is soluble at a pH of about 1 to about 5.

64. The use or composition as claimed in any of claims 46-63 wherein the difference in Hansen solubility parameter between active pharmaceutical ingredient and the combination of the first polymer and the second polymer is less than 10 MPa^{1/2}.

65. The use or composition as claimed in claim 64 wherein the difference in Hansen solubility parameter between active pharmaceutical ingredient and the combination of the first polymer and the second polymer is less than 7 MPa^{1/2}.

66. The use or composition as claimed in any of claims 46-65 wherein the solidified melt extrudate comprises an NSAID in an amorphous form and a combination of dimethylaminoethyl methacrylate co-polymer and polyvinylpyrrolidone-vinyl acetate co-polymer (PVPVA64) wherein the extrudate comprises from 30%-45% by weight NSAID wherein the extrudate is prepared using a hot melt extrusion process at a temperature from about 75° C. to about 95° C.

67. The use or composition as claimed in claim 66 wherein the weight ratio of the dimethylaminoethyl methacrylate co-polymer and polyvinylpyrrolidone-vinyl acetate co-polymer (PVPVA64) is from 5:1 to 15:1.

68. The use or composition as claimed in claim 67 wherein the weight ratio of the NSAID, the dimethylaminoethyl methacrylate co-polymer and the polyvinylpyrrolidone-vinyl acetate co-polymer (PVPVA64) is from 5:8:1 to 10:15:1.

69. The use or composition as claimed in any of claims 46-68 wherein the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64).

70. The use or composition as claimed in claim 69 wherein the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate is prepared using a hot melt extrusion process at a temperature from about 75° C. to about 95° C.

71. The use or composition as claimed in claim 70 wherein the solidified melt extrudate comprises 30%-45% by weight of an NSAID in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate is prepared using a hot melt extrusion process at a temperature from about 75° C. to about 95° C. and wherein the weight ratio of the NSAID, the dimethylaminoethyl methacrylate co-polymer and the polyvinylpyrrolidone-vinyl acetate co-polymer (PVPVA64) is from about 5:8:1 to about 10:15:1.

72. The use or composition as claimed in any of claims 46-48 wherein the solidified melt extrudate comprises ibuprofen in an amorphous form and a combination of a first polymer having a glass transition temperature of between 40° C. and 50° C. and a second polymer having a glass transition temperature of between 90° C. and 110° C. wherein the extrudate comprises from 30%-45% by weight of ibuprofen.

73. The use or composition as claimed in claim 72 wherein when the second polymer of the combination of two polymers is selected to be a homopolymer it has an average molecular weight of 1,000 to 5,000 Daltons and when the second polymer of the combination of two polymers is selected to be a co-polymer it has an average molecular weight of from 45,000 to 70,000 Daltons.

74. The use or composition as claimed in claim 72 or claim 73 wherein the weight ratio of the of the first polymer and the second polymer is from 5:1 to 15:1.

75. The use or composition as claimed in claim 72 or claim 73 wherein the weight ratio of the ibuprofen, the first polymer and the second polymer is from 5:8:1 to 10:15:1.

76. The use or composition as claimed in claim 72 or claim 73 wherein the solidified melt extrudate comprises 30%-45% by weight of ibuprofen in an amorphous form and a combination of 45%-65% by weight of the first polymer and 1%-10% by weight of the second polymer.

77. The use or composition as claimed in claim 76 wherein the solidified melt extrudate comprises 30%-45% by weight of ibuprofen in an amorphous form and a combination of 45%-65% by weight of the first polymer and 1%-10% by weight of the second polymer wherein the extrudate is prepared using a hot melt extrusion process at a temperature from about 75° C. to about 95° C.

78. The use or composition as claimed in claim 77 wherein the solidified melt extrudate comprises 30%-45% by weight of ibuprofen in an amorphous form and a combination of 45%-65% by weight of the first polymer and 3%-8% by weight of the second polymer wherein the extrudate is prepared using a hot melt extrusion process at a temperature from about 75° C. to about 95° C. and wherein the weight ratio of the ibuprofen, the first polymer and the second polymer is from about 5:8:1 to about 10:15:1.

79. The use or composition as claimed in claim 78 wherein the solidified melt extrudate comprises 30%-45% by weight of ibuprofen in an amorphous form and a combination of 45%-65% by weight of the first polymer and 1%-10% by weight of the second polymer wherein the extrudate is prepared using a hot melt extrusion process at a temperature from about 75° C. to about 95° C. and wherein when the second polymer of the combination of two polymers is selected to be a homopolymer it has an average molecular weight of 1,000 to 5, 000 Daltons and when the second polymer of the combination of two polymer is selected to be a co-polymer it has an average molecular weight of from 45,000 to 70,000 Daltons.

80. The use or composition as claimed in claim 72 wherein the first polymer of the combination of two polymers is selected from polymethacrylates including but not limited to amino methacrylate copolymer, amino-methyl methacrylate, methacrylic acid methyl methacrylate, dimethylaminoethyl methacrylate co-polymer.

81. The use or composition as claimed in claim 80 wherein the second polymer of the combination of two polymers is selected from polyvinylpyrrolidone, polyvinylpyrrolidone-vinyl acetate co-polymer and polyvinyl caprolactam-polyvinyl acetate-polyethylene glycol graft copolymer.

82. The use or composition as claimed in any of claims 73-81 wherein the combination of polymers is soluble at a pH of about 1 to about 5.

83. The use or composition as claimed in any of claims 73-82 wherein the difference in Hansen solubility parameter between active pharmaceutical ingredient and the combination of the first polymer and the second polymer is less than 10 MPa^{1/2}.

84. The use or composition as claimed in claim 83 wherein the difference in Hansen solubility parameter between active pharmaceutical ingredient and the combination of the first polymer and the second polymer is less than 7 MPa^{1/2}.

85. The use or composition as claimed in claim 72 wherein the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate comprises from 30%-45% by weight of ibuprofen wherein the extrudate is prepared using a hot melt extrusion process at a temperature from about 75° C. to about 95° C.

86. The use or composition as claimed in claim 85 wherein the weight ratio of the dimethylaminoethyl methacrylate co-polymer and polyvinylpyrrolidone-vinyl acetate co-polymer (PVPVA64) is from 5:1 to 15:1.

87. The use or composition as claimed in claim 85 wherein the weight ratio of the ibuprofen, the dimethylaminoethyl methacrylate co-polymer and the polyvinylpyrrolidone-vinyl acetate co-polymer (PVPVA64) is from 5:8:1 to 10:15:1.

88. The use or composition as claimed in claim 72 wherein the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64).

89. The use or composition as claimed in claim 88 wherein the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64) wherein the extrudate is prepared using a hot melt extrusion process at a temperature from about 75° C. to about 95° C.

90. The use or composition as claimed in claim 89 wherein the solidified melt extrudate comprises 30%-45% by weight of ibuprofen in an amorphous form and a combination of 45%-65% by weight of dimethylaminoethyl methacrylate co-polymer and 3%-8% by weight of polyvinylpyrrolidone-vinyl acetate co-polymer (PVPVA64) wherein the extrudate is prepared using a hot melt extrusion process at a temperature from about 75° C. to about 95° C. and wherein the weight ratio of the ibuprofen, the dimethylaminoethyl methacrylate co-polymer and the polyvinylpyrrolidone-vinyl acetate co-polymer (PVPVA64) is from about 5:8:1 to about 10:15:1.
